(19) United States
(12) Patent Application Publication
(10) Pub. No.: US 2016/0045634 A1
(43) Pub. Date: Feb. 18, 2016

(54) Analgesic Formulation and Delivery Bandage

(71) Applicant: Wayne Caleb Williams, Brandon, FL (US)

(72) Inventor: Wayne Caleb Williams, Brandon, FL (US)

(21) Appl. No.: 14/460,411

(22) Filed: Aug. 15, 2014

(51) Int. Cl. A61L 15/44; A61K 36/61; A61K 33/24; A61K 36/14; A61F 13/02; A61K 36/15; A61K 35/02; A61K 33/08; A61F 13/00; A61K 31/05; A61K 36/45

(52) U.S. Cl. CPC A61L 15/44; A61K 31/05; A61K 36/61; A61K 33/24; A61K 36/14; A61K 36/45; A61K 36/15; A61K 35/02; A61K 33/08; A61F 13/00029; A61F 13/025; A61F 13/0236; A61L 2300/402; A61L 2300/45; A61L 2300/606; A61F 2013/00089

(57) ABSTRACT

A medical dressing and analgesic formulation that is operable to provide pain relief to an injured area of the human body wherein the medical dressing is constructed so as to allow a user to create a customized size for each application of the medical dressing. The medical dressing further includes a body being planar in manner having a plurality of contiguous portions that are separated by a perforation. Each contiguous portion includes a medicament layer thereon that is operable to treat an injured are for pain. The contiguous portions in one embodiment of the medical dressing are further configured to be divisible into four quadrants wherein the user can select as few as one quadrant or more than one quadrant of each continuous portion so as to size the medical dressing to the appropriate size needed to treat the desired area of the body.

### FIELD OF THE INVENTION

The present invention relates generally to analgesic formulations and medical dressings, more specifically but not by way of limitation a bandage or a patch that is manufactured so as to allow a user to customize the size of the dressing applied to an injured area and wherein the dressing is impregnated with an analgesic formulation operable to provide relief to the applied injured area.

### BACKGROUND

Millions of individuals every year purchase over the counter and prescription medicines for relief of pain. Whether it is for chronic arthritis or for a temporary injury such as but not limited to a sprained joint, it is common to utilize pain relief of some variety. For more serious injuries, health professionals will prescribe prescription painkillers and the like to help alleviate the pain for the patient. For more routine injuries such as but not limited to sprains, muscle aches and other similar maladies, it is common for individuals to take oral analgesics or apply a topical analgesic to the affected area. Topical analgesics are available in many forms such as but not limited to sprays or ointments and are applied by the user as necessary for the treatment of pain.

Another type of available treatment are pain patches. Pain patches typically consist of a patch of a certain size that includes an analgesic associated therewith for the topical application of the analgesic. Pain patches are typically offered in a few sizes configured for different areas of the human body. For example, larger rectangular patches are commercially available that are designed to be applied to a user's lower back for the treatment of back pain. One issue with conventionally available pain patches is their availability in only a few select sizes. Conventional pain patches are available typically only in three sizes, which can create difficulties for the user depending upon their application and body size.

Another issue with conventionally available patches is the time period in which a patch can be applied to an injured area. Most often the medicinal compound delivered by a patch or bandage contains substances, which can cause skin irritation. These substances must be applied for a limited time only so as to reduce the possible side effects. A compound comprised of naturally occurring compounds would be an improvement allowing a longer application to an injured area without the effect of skin irritation.

Accordingly, there is a need for a medical dressing in the form of a patch or a bandage that is manufactured so as to facilitate customized sizing by the patient and wherein the medical dressing includes an analgesic formulation containing ingredients operable to expedite the healing of the injured area.

### SUMMARY OF THE INVENTION

It is the object of the present invention to provide a medical dressing in the form of a patch and/or bandage wherein the medical dressing is manufactured so as to allow a user to customize the size thereof.

Another object of the present invention is to provide a medical dressing configured to be sized by the patient wherein the medical dressing is operable to deliver an analgesic formulation operable to relieve pain.

A further object of the present invention is to provide a medical dressing that is configurable in size impregnated with a topical analgesic wherein in one embodiment thereof the medical dressing includes a base layer, medicament layer and protective layer of material.

Yet another object of the present invention is to provide a medical dressing operable to treat pain wherein in one embodiment the medical dressing is manufactured in a rolled form.

An additional object of the present invention is to provide a medical dressing operable to provide pain relief wherein one embodiment is manufactured in an integrated pre-sized form having a quadrant of smaller available sizes therein facilitating the ability for a user to customize the size of the medical dressing.

Still a further object of the present invention is to provide a medical dressing operable to provide pain relief for an injured area wherein one embodiment includes a base layer manufactured of breathable fabric so as to allow a longer application thereof.

Another object of the present invention is to provide a medical dressing wherein the size thereof is configurable by a patient so as to apply a pain relief compound to a desired portion of their body wherein one embodiment is further operable to provide compression.

A further object of the present invention is to provide an analgesic formulation infused into a medical dressing that is configurable in size wherein the analgesic formulation is operable to expedite the healing process of the injured area.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a bottom view of an embodiment of the present invention; and

FIG. 2 is a bottom view of an alternative embodiment of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated a medical dressing 100 constructed according to the principles of the present invention.

The present invention includes a formulation that is pine resin based wherein the formulation has improved transdermal properties. The formulation of the present invention has a preferred formula of from about 5% wt to about 75% wt of Pine Resin; 0.1% wt to about 10% wt of polyacrylic acid; 5% wt to about 80% wt glycerin and 1.25% wt to about 16% wt menthol. The aforementioned formulation and ranges comprise a preferred formulation base. It is further contemplated within the scope of the present invention that the formulation can additionally be comprised of: 0.5% wt to about 5% wt titanium; 0.5% wt to about 5% wt kaolin; 0.01% wt to about 1.2% wt aluminum hydroxide; 60% wt to about 72% wt water; 0.01% wt to about 1% wt eucalyptus; 0.01% wt to about 1% wt juniper berry and 0.01% wt to about 1% wt wintergreen. The aforementioned formulation is impregnated into the medical dressing 100 of the present invention in order to facilitate transdermal delivery. While in the preferred embodiment the formulation of the present invention is impregnated into the medical dressing 100, it is further contemplated within the scope of the present invention that the formulation be manufactured as a gel, lotion, spray or enhanced oil.

Referring in particular herein to FIG. 2, an embodiment 99 of the medical dressing 100 is illustrated therein. The medical dressing 100 includes a plurality of portions 5 (only 2 are illustrated herein) forming a body 10 that is planar in manner and generally rectangular in shape. The body 10 is manufactured in a continuous manner having a plurality of portions 5 that can be packaged in either a rolled form or in some similar desirable packaging. The portions 5 are operably coupled at ends 6, 7 utilizing a perforation 8. The perforations facilitate the separation of a portion 5 from its contiguous adjacent portion and further can facilitate the dispensal of at least two adjacent portions 5 if desired by the user. The body 10 includes a base layer 15 that is manufactured from a non-woven, cohesive stretchable and water resistant fabric. The base layer 15 is further operable to be manufactured to be non-absorbent so as to resists absorption of the medicament layer 20 discussed further herein. The base layer 15 functions to provide a source of compression subsequent the embodiment 99 being applied to the user. It is contemplated within the scope of the present invention that the base layer 15 could be treated with the appropriate chemicals to produce the desired results of water resistance and non-absorbency. Those skilled in the art will recognize that numerous types of compounds and/or techniques could be utilized to produce the desired results of non-absorbency and/or water resistance for the base layer 15.

Superposed on the inner surface 13 of the base layer 15 is the medicament layer 20. The medicament layer 20 consists of a gel material having the analgesic formulation previously disclosed herein. While Table 1 herein identifies a preferred embodiment of specific wt percentages for each of the ingredients of the formulation comprising the medicament layer 20, it is contemplated within the scope of the present invention that the formulation can vary within the wt percentage ranges disclosed herein. The medicament layer 20 is superposed substantially across the inner surface 13 of each portion 5 of the embodiment 99. As is illustrated herein, it is contemplated within the scope of the present that a void 17 is present around the medicament layer 20 so as to allow for dispersal of the medicament layer 20 as a result of any compression force applied thereto during application of the embodiment 99 to the user. While no particular void is required, good results have been achieved utilizing a void 17 that is approximately five millimeters in width. Superposed the medicament layer 20 is protective layer 30. The protective layer 30 functions to protect the medicament layer 20 from dispersal until the embodiment 99 is applied to the user. The protective layer 30 is releasably superposed each portion 5 of the body 10 in a continuous manner sharing perforation 8. As a user removes a portion 5 from the body 10 the protective layer 30 remains superposed the removed portion 5. Prior to the application of the embodiment 99, the protective layer 30 is removed so as to expose the medicament layer 20 in order to facilitate application to the desired area by the user. It is contemplated within the scope of the present invention that the protective layer 30 could be manufactured from numerous suitable materials such as but not limited to plastic film. Each portion 5 is sized to be approximately ten centimeters in width and thirty-two centimeters in length. Utilizing this size and manufacturing the body 10 with contiguous portions 5 provides sizing of the embodiment 99 of the medical dressing 100 that is desirable for treating wounds such as but not limited to sprains or joint pain. The aforementioned size and the contiguous portions 5 facilitate coverage of the injured area not available with conventional bandages as the embodiment allows a user to size the embodiment 99 to a required size specific to each user's body area and size.

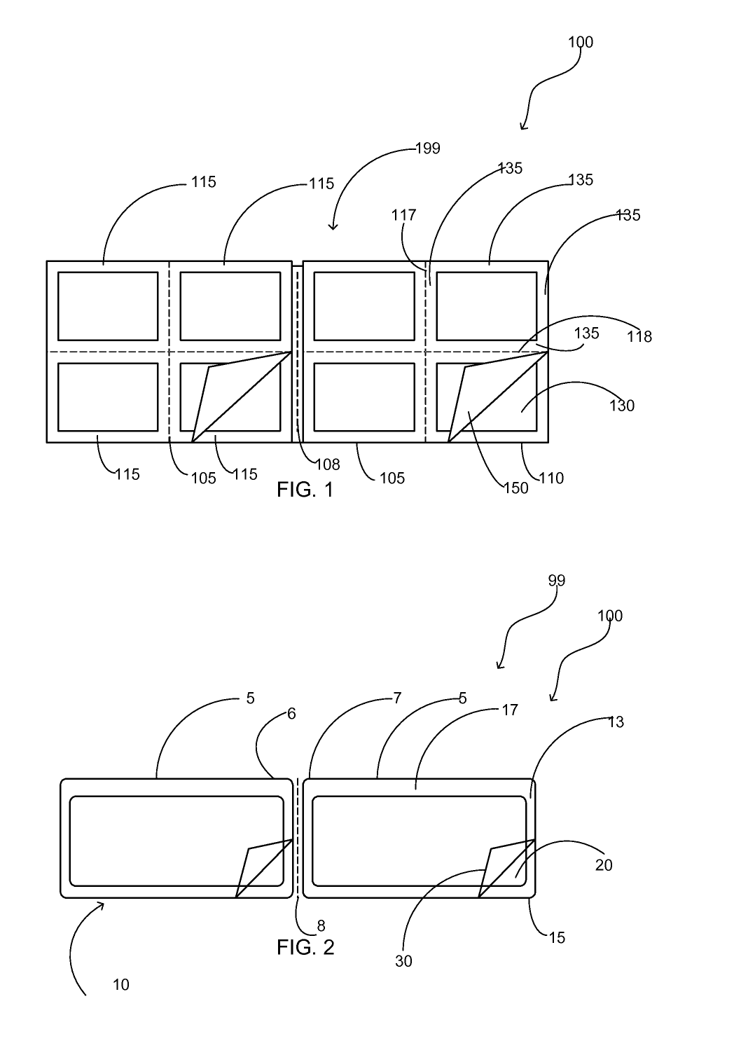

Referring in particular to FIG. 1 herein, a second embodiment 199 of the medical dressing 100 is illustrated therein. Embodiment 199 includes body 110 that is planar in manner and rectangular in shape. The body 110 includes portions 105 that are contiguous having a perforation 108 therebetween that facilitates separation of the portions 105 by a user. Each portion 105 is further divided in quadrants 115 wherein the quadrants 115 are joined along perforation edges 117,118. The body 110 includes a base layer 120 that is manufactured from a breathable textile fabric having at least some elasticity. The breathable fabric of the base layer 120 facilitates the prolonged application of the embodiment 199 wherein the breathability substantially reduces skin irritation caused by conventional bandage material. The material of the base layer 120 permits application by the user for at least twelve hours without any skin irritation to the user. The base layer 120 has superposed thereon using appropriate techniques a medicament layer 130. The medicament layer 130 is a gel material that is comprised of the components referenced herein in Table 1. The medicament layer 130 is operable to treat an area of the user's body suffering from either chronic pain or an injury such as but not limited to a sprain. The medicament layer 130 is superposed on each quadrant 115. While Table 1 herein identifies a preferred embodiment of specific wt percentages for each of the ingredients of the formulation comprising the medicament layer 130, it is contemplated within the scope of the present invention that the formulation can vary within the wt percentage ranges disclosed herein.

Surrounding the medicament layer 130 on each quadrant 115 is adhesive border 135. The adhesive border 135 is manufactured from suitable adhesive and is operable to releasably secure the embodiment 199 to the user. The adhesive border 135 in its desired embodiment is only one to three millimeters in width. This width provides sufficient adhesion to the desired area of the user and further facilitates application of the medicament layer 130 to a larger area of the body wherein the user will utilize more than one quadrant 115 of the body 110. The quadrants 115 of the body 110 facilitate the customization of the size of the embodiment 199 that is desired and/or needed by the user to treat a specific injured area. As the body 110 is manufactured having perforations 108 and perforation edges 117, 118 this allows the user to remove either a single quadrant 115 or more than one portion 105 so as to cover the injured area as needed. Utilizing an adhesive border 135 of the aforementioned width provides sufficient adhesion and also serves to minimize the reduction of application of medicament layer 130 to an injured area upon the utilization of more than one quadrant 115. While the body 110 is illustrated herein as having perforation edges 117,118 forming quadrants 115, it is contemplated within the scope of the present invention that each portion 105 could be manufactured so as to have either more or less perforations edges 117,118 creating a portion 105 having more than four sub-portions.

Embodiment 199 further includes a protective layer 150. The protective layer 150 is releasably secured to the body 110 utilizing suitable techniques and is operable to protect the medicament layer 130. The protective layer 150 shares the perforation edges 117, 118 and perforation 108. As the body 110 is sized by the user, the protective layer 150 remains releasably secured to each quadrant 115. Immediately prior to application of the embodiment 199, the user will remove the protective layer 150 so as to expose the medicament layer 130. While numerous types of material could be utilized to manufacture the protective layer 150, good results have been achieved utilizing a plastic film for the protective layer 150.

Each portion 105 of the body 110 is approximately ten centimeters in width and fourteen centimeters in length. As previously discussed herein the portions 105 are equally divisible into quadrants 115. This technique and the size disclosed herein facilitates the customization of size of the embodiment 199 facilitating the use of the correct size of medical dressing 100 that is required so as to either eliminate waste of the medical dressing 100 and/or enable a user to treat an area that is too large for a conventional bandage.

The formulation for the medicament layer 20, 130 disclosed herein for embodiments 99, 199 is operable to treat both chronic pain conditions as well as acute injuries such as but not limited to sprains and other muscle injuries. Efficacy results utilizing the medicament layer 20, 130 include but are not limited to the following: 85 percent of chronic pain sufferers experienced pain relief during use; 100% of acute pain sufferers experienced relief within twelve hours of application of the medicament layer 20, 130; 70% of chronic pain sufferers experienced greater relief of symptoms within 24 hours.

TABLE 1

| Components | Wt. Percentage |
|---|---|
| Menthol | 1.25% |
| Wintergreen | 0.05% |
| Juniper Berry | 0.05% |
| Eucalyptus | 0.05% |
| Polyacrylic Acid | 5% |
| Kaolin | 1% |
| Titanium | 1% |
| Glycerin | 20% |
| Aluminum hydroxide | 0.20% |
| Water | 61.40% |
| Pine Resin | 10% |

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An analgesic formulation comprising:
Menthol, wherein the menthol is from about 1.25% wt to about 16% wt;
Polyacrylic acid, wherein the polyacrylic acid is from about 0.1% wt to about 10% wt; and
Glycerin, wherein the glycerin is from about 5% wt to about 80% wt.

2. The analgesic formulation as recited in claim 1, and further comprising from 5% wt to about 75% of Pine Resin and 0.01% wt and to about 1% wt of eucalyptus, juniper berry and wintergreen.

3. The analgesic formulation as recited in claim 2, and further comprising from about 0.5% wt to about 5% wt titanium.

4. The analgesic formulation as recited in claim 3, and further comprising from about 0.5% wt to about 5% wt kaolin.

5. The analgesic formulation as recited in claim 4, and further comprising from about 0.01% wt to about 1.2% wt aluminum hydroxide.

6. The analgesic formulation as recited in claim 5, and further comprising from about 60% wt to about 72% wt water.

7. The analgesic formulation as recited in claim 6, wherein the analgesic formulation is prepared in a form selected from one of the following forms consisting of: a gel, a lotion, a spray or an enhanced oil.

8. A medical dressing operable to be superposed an injured area of a human body for the purpose of pain relief comprising:
a body, said body being planar in manner and rectangular in shape, said body having a plurality of contiguous portions, each of said plurality of contiguous portions having a perforation intermediate thereto, said body having a base layer, said base layer having a first surface and a second surface, each of said plurality of contiguous portions having a first end and a second end, each of said plurality of contiguous portions having an upper edge and a lower edge,
a medicament layer, said medicament layer being superposed said first surface of said base layer;
a protective film, said protective film being releasably secured to first surface of said base layer superposed said medicament layer; and
wherein each of said plurality of contiguous portions further include one horizontal perforation edge and one vertical perforation edge so as to equally divide each of said plurality of contiguous portions into four quadrants so as to facilitate the selection of a customized size of the medical dressing by a user.

9. The medical dressing as recited in claim 8, and further including an adhesive border, said adhesive border extending circumferentially around each of said four quadrants of said plurality of contiguous portions.

10. The medical dressing as recited in claim 9, wherein said base layer is manufactured from a breathable textile material.

11. The medical dressing as recited in claim 10, wherein said medicament layer includes the following materials: Menthol, wintergreen oil, Juniper berry, Eucalyptus, Polyacrylic acid, Kaolin, Titanium, Glycerin, Aluminum hydroxide and Water.

12. The medical dressing as recited in claim 11, wherein each of said contiguous portions of said body are approximately 10 centimeters in width and 14 centimeters in length.

13. The medical dressing as recited in claim 12, and further including pine resin, wherein the pine resin of said medicament layer is present from about 5% wt to about 75% wt in each of said four quadrants of said plurality of contiguous portions.

14. The medical dressing as recited in claim 13, wherein the menthol is from about 1.25% wt to about 16% wt in each of said four quadrants of said plurality of contiguous portions.

15. A medical dressing including a medicament that is operable to provide pain relief to an area of a human body wherein the medical dressing is further operable to apply compression comprising:
a body, said body being planar in manner and rectangular in shape, said body being manufactured from a stretchable material, said body comprising of a plurality of contiguous portions, said body having an upper edge and a lower edge, said plurality of contiguous portions having a first end and a second end, said body further including a perforation intermediate each of said plurality of contiguous portions, said plurality of contiguous portions having a first surface and a second surface;
a medicament layer, said medicament layer being a gel, said medicament layer being superposed said first surface of each of said plurality of contiguous portions, said medicament layer operable to provide pain relief; and
a protective layer, said protective layer being superposed said medicament layer, said protective layer being releasably secured, said protective layer being a plastic film; and
wherein said body being formed in a rolled manner and operable to allow a user to disconnect at least one of said plurality of contiguous portions so as to provide a user with a medical dressing of an appropriate size required by a user.

16. The medical dressing as recited in claim 15, wherein said plurality of contiguous portions are approximately 10 centimeters in width by thirty two centimeters in length.

17. The medical dressing as recited in claim 16, wherein said medicament layer is formulated with the following components wherein the components are within referenced weight percentages: Pine resin, wherein the pine resin is from about 5% wt to about 75% wt; menthol, wherein the menthol is from about 1.25% wt to about 16% wt; polyacrylic acid, wherein the polyacrylic acid is from about 0.1% wt to about 10% wt; and glycerin, wherein the glycerin is from about 5% wt to about 80% wt.

18. The medical dressing as recited in claim 17, and further including pine resin, wherein the pine resin is from about 5% wt to about 75% wt and wherein the menthol of the medicament layer is from about 1.25% wt to about 16% wt in each of said plurality of contiguous portions of said body.

19. The medical dressing as recited in claim 18, wherein the medicament layer further includes from about 0.01% wt to about 1% wt of eucalyptus, juniper berry and wintergreen; from about 0.5% wt to about 5% wt titanium, from about 0.5% wt to about 5% wt kaolin, from about 0.01% wt to about 1.2% wt aluminum hydroxide and from about 60% wt to about 72% wt water.

20. The medical dressing as recited in claim 19, wherein the medicament layer has a surface area that extends approximately 9 centimeters in width and 30.5 centimeters in length.
